(19) United States
(12) Patent Application Publication
(10) Pub. No.: US 2006/0211069 A1
(43) Pub. Date: Sep. 21, 2006

(54) Device including a proteinaceous factor, a recombinant proteinaceous factor, and a nucleotide sequence encoding the proteinaceous factor

(75) Inventor: John W. Allen, Greensboro, NC (US)

Correspondence Address: SMITH MOORE LLP, P.O. BOX 21927, GREENSBORO, NC, 27420, US

(73) Assignee: North Carolina A&T State University

(21) Appl. No.: 11/437,009

(22) Filed: May 18, 2006

Related U.S. Application Data
(62) Division of application No. 10/410,649, filed on Apr. 9, 2003.

(51) Int. Cl. G01N 33/53; C12M 1/34

(52) U.S. Cl. 435/7.92; 435/287.2

(57) ABSTRACT

A device that includes a proteinaceous factor is disclosed. The proteinaceous factor is encoded by the nucleotide sequence of any one of SEQ ID NO.: 1, a degenerate variant of SEQ ID NO.: 1, and a complement of SEQ ID NO.: 1. The proteinaceous factor may be a recombinant. In addition, the device may include any one of (i) Immunoglobulin G (IgG) bound non-specially to the proteinaceous factor, (ii) at least one diagnostic label bound to the proteinaceous factor, (iii) Immunoglobulin G bound non-specially to the proteinaceous factor and at least one diagnostic label bound to the proteinaceous factor, and (iv) at least one base supporting the proteinaceous factor.

### BACKGROUND OF THE INVENTION

(1) Field of the Invention

The present invention relates generally to a device including a proteinaceous factor, a recombinant proteinaceous factor, and a nucleotide sequence encoding a proteinaceous factor.

(2) Description of the Prior Art

The first line of defense when fighting an infectious disease is the host's immune system. An understanding of how this system works can lead to an understanding of how infectious diseases function. When a disease is introduced into an animal, the disease triggers specific immune responses, often in the form of antibodies. Studies of the immune system can also lead to valuable analyses of why the immune system is or is not effective in fighting the disease.

Thus, there remains a need for a new and improved device that includes a proteinaceous factor while, at the same time there remains a need for a recombinant of the proteinaceous factor and a nucleotide sequence encoding the proteinaceous factor.

### SUMMARY OF THE INVENTION

The present invention is directed to a device that includes a proteinaceous factor encoded by the nucleotide sequence of any one of SEQ ID NO.: 1, a degenerate variant of SEQ ID NO.: 1, and a complement of SEQ ID NO.: 1. The proteinaceous factor may be a recombinant. In addition, the device may include any one of (i) Immunoglobulin G (IgG) bound non-specially to the proteinaceous factor, (ii) at least one diagnostic label bound to the proteinaceous factor, (iii) Immunoglobulin G bound non-specially to the proteinaceous factor and at least one diagnostic label bound to the proteinaceous factor, and (iv) at least one base supporting the proteinaceous factor.

Non-limiting examples of the types of devices contemplated by the applicant of the present invention include any one of an enzyme immuno assay, an electro-immuno blot, a dot blot, an antibody isolator, an antibody purifier, an antibody isolator and purifier.

The base may be an inert solid support. Such inert solid support may include a plurality of wells. Non-limiting examples of inert solid supports include any one of a polymer, a glass, a paper and combination thereof. Non-limiting examples of an inert solid support useable as a base for an enzyme immuno assay includes polymers, such as a polystyrene (PS), polyethylene (PE) that may include low-density polyethylene (LDPE) and high-density polyethylene (HDPE), a polypropylene (PP), polyethylene terephthalate (PET), and polyethylene terephthalate glycolate (PETG). Non-limiting examples of inert solid supports useable as a base for any one of an electro-immuno blot and a dot blot include a cellulosic membrane such as a nitrocellulose. Non- limiting examples of inert solid supports useable as a base for any one of an antibody isolator; an antibody purifier; and antibody isolator and purifier include microbeads and a porous membrane supporting the proteinaceous factor.

In an embodiment, the device is for detecting any one of monoclonal mammalian IgG, polyclonal mammalian IgG, and monoclonal mammalian IgG, polyclonal mammalian IgG by binding to any one thereof. The mammalian IgG may be human IgG such as any one of human IgG₁, human IgG₂, human IgG₃, human IgG₄ and combination thereof. Alternatively, the mammalian IgG may be any one of horse IgG, bovine IgG, rat IgG, swine IgG, mouse IgG, sheep IgG, goat IgG, guinea pig IgG, hamster IgG, and combinations thereof.

In a device including Immunoglobulin G (IgG) bound non-specially to the proteinaceous factor, the Immunoglobulin G (IgG) is selected for its antigenic specificity. Non-limiting examples of such antigenic specific Immunoglobulin G (IgG) include any one of human IgG, which may be any one of human IgG₁, human IgG₂, human IgG₃, human IgG4 and combination thereof, horse IgG, bovine IgG, rat IgG, swine IgG, mouse IgG, sheep IgG, goat IgG, guinea pig IgG, hamster IgG, and combinations thereof.

Non-limiting examples of a diagnostic label include any one of ferritin, gold, silver, a chemical conjugate, a radioactive component and combination thereof. One non-limiting example of a chemical conjugate is an enzyme conjugate. Non-limiting examples of enzyme conjugates include one of a horseradish peroxidase (HRP), an alkaline phosphatase (APAAP), a lactoperoxidase (LPO), a glucose oxidase, digoxigenin and combinations thereof. The enzyme conjugate may then act on a substrate, such as a dye substrate or a chemiluscent substrate. A non-limiting example of a chemiluminescent component is a chemiluminescent substrate for alkaline phosphatase (APAAP) detection. Non-limiting examples of diagnostic labels including a radioactive component may include any one of ³³P, ³H, ³⁵S, ¹²¹I, ¹³¹I, ³²P, ⁵⁴CR, and combination thereof.

An N-terminal amino acid sequence of the proteinaceous factor is alanine, proline, threonine, valine, proline, glutamine, alanine, proline, alanine, threonine, glycine, glutamine, glutamine, alanine, alanine, glutamic acid, valine, threonine, glutamic acid, leucine, lysine, aspartic acid, valine, lysine, phenylalanine, threonine, phenylalanine, lysine, and methionine. Bases 103 through 189 of SEQ ID NO.: 1 encode this N-terminal amino acid sequence. A computer-generated translation of the SEQ ID NO.: 1 is SEQ ID NO.: 2. Thus, the N-terminal amino acid sequence corresponds substantially to amino acids 35 through 63 of SEQ ID NO.: 2. In addition, a native N-terminal amino acid sequence is substantially the same as an N-terminal amino acid sequence of the recombinant proteinaceous factor. Thus, the N-terminal amino acid sequence of the proteinaceous factor conforms substantially to the corresponding N-terminal amino acid sequence of the computer generated translation of the nucleotide sequence.

The present invention contemplates a nucleotide sequence at least about 80% identical to SEQ ID NO.: 1. Also contemplated is a nucleotide sequence that encodes a proteinaceous factor having an amino acid sequence that is at least about 80% identical to SEQ ID NO.: 2. In addition, it is contemplated that the nucleotide sequence encodes a proteinaceous factor having any one of the sequence of SEQ ID NO.: 2 and SEQ ID NO.: 2 with conservative amino acid substitutions. Likewise, it is contemplated that the nucleotide may encode a proteinaceous factor comprising any one of the amino acid sequence of SEQ ID NO.: 2 and a fragment of SEQ ID NO.: 2 at least 8 residues in length.

The proteinaceous factor of the present invention is a receptor for the F_{C} region of mammalian IgG. The mammalian IgG may be human IgG such as any one of human IgG1, human IgG2, human IgG3, human IgG4 and combination thereof. Alternatively, the mammalian IgG may be any one of horse IgG, bovine IgG, rat IgG, swine IgG, mouse IgG, sheep IgG, goat IgG, guinea pig IgG, hamster IgG, and combinations thereof. In addition, the proteinaceous factor of the present invention has a molecular weight of about 96,000 Daltons as measured using a non-denaturing gel. Another example of a receptor is human albumin.

In an embodiment, the proteinaceous factor of the present invention is a recombinant. That is, a nucleotide sequence of any one of SEQ ID NO.: 1, a degenerate variant of SEQ ID NO.: 1, and a complement of SEQ ID NO.: I is inserted into a plasmid vector. The plasmid vector is translated into a host cell and the host cell expresses the proteinaceous factor.

The nucleotide sequence of the present invention is the nucleotide sequence in competent E. coli host cells of NRRL Deposit No.: B-30634. An nucleotide sequence isolated from the cell in ATCC Deposit No. 55195 is at least 90% identical to an nucleotide sequence isolated from the competent E. coli host cells of NRRL Deposit No.: B-30634 and the percent identity is calculated using FASTDB with the parameters set such that percentage of identity is calculated over the full length of the reference nucleotide. Gaps in homology of up to 5% of the total number of nucleic acids in the reference nucleotide sequence are allowed. The isolated nucleotide sequence in ATCC Deposit No. 55195 is at least 95% identical to the isolated nucleotide sequence in NRRL Deposit No.: B-30634.

The isolated nucleic acid is the nucleotide sequence of SEQ ID NO.: 1, or a degenerate variant of SEQ ID NO.: 1. The isolated DNA of the nucleotide sequence may consist of SEQ ID NO.: 1. The isolated nucleic acid encodes a proteinaceous factor with the amino acid sequence of SEQ ID NO.: 2. The isolated nucleic acid sequence hybridizes under highly stringent conditions to a hybridization probe and consists of SEQ ID NO.: 1, or the complement of SEQ ID NO.: 1.

A resultant isolated nucleic acid is at least about 80% identical to SEQ ID NO.: 1. Also, a resultant isolated nucleic acid encodes a proteinaceous factor, the amino acid sequence of which is at least 80% identical to SEQ ID NO.: 2. In addition, resultant isolated nucleic acid encodes a proteinaceous factor having the sequence of SEQ ID NO.: 2, or SEQ ID NO.: 2 with conservative amino acid substitutions, or of a fragment of SEQ ID NO.: 2 at least 8 residues in length.

Another way of looking at an embodiment of the invention is as a DNA sequence, which comprises SEQ ID NO.: I operably linked to a heterologous coding sequence. Yet another way of looking at an embodiment of the invention is as an expression vector comprising the nucleic acid of SEQ ID NO.: 1 operably linked to an expression control sequence.

Another embodiment of the invention involves a cultured cell comprising an expression vector comprising the nucleic acid of SEQ ID NO.: 1. The cultured cell contains the nucleic acid of SEQ ID NO.: 1, operably linked to an expression control sequence. Alternatively, the invention involves a cultured cell comprising a maintenance vector comprising the nucleic acid of SEQ ID NO.: 1. The cultured cell contains the nucleic acid of SEQ ID NO.: 1, operably linked in a manner that preserves the sequence for future transformation into a cell to operably link the sequence to an expression control sequence. In this manner, a cultured cell transfected with the vector, or a progeny of the cell may be used to express the proteinaceous factor.

In an embodiment, a proteinaceous factor results from culturing cell containing the nucleic acid of SEQ ID NO.: 1, operably linked to an expression control sequence under conditions permitting expression of the proteinaceous factor. That is culturing the cell(s) under conditions that permit expression under the control of the expression control sequence, and purifying the proteinaceous factor from the cell or the medium of the cell produce the proteinaceous factor.

Yet another way of looking at the invention is as a single-stranded nucleic acid that hybridizes under highly stringent conditions to a nucleic acid having the sequence of SEQ ID NO.: 1. Even another way of looking at the invention is as an isolated nucleic acid comprising at least 10 consecutive nucleotides of the complement of SEQ ID NO.: 1. Still another way of looking at the invention is as a purified proteinaceous factor, the amino acid sequence of which consists of SEQ ID NO.: 2.

Accordingly, one aspect of the present invention is to provide a device including a proteinaceous factor encoded by any one of SEQ ID NO.: 1, a degenerate variant of SEQ ID NO.: 1, and a complement of SEQ ID NO.: 1.

Another aspect of the present invention is to provide a recombinant proteinaceous factors encoded by any one of SEQ ID NO.: 1, a degenerate variant of SEQ ID NO.: 1, and a complement of SEQ ID NO.: 1. The recombinant proteinaceous factor useable in a devise or alone.

Still another aspect of the present invention is to provide a device that includes proteinaceous factor encoded by the nucleotide sequence of any one of SEQ ID NO.: 1, a degenerate variant of SEQ ID NO.: 1, and a complement of SEQ ID NO.: 1. The proteinaceous factor is a recombinant. In addition, the device may include any one of (i) Immunoglobulin G (IgG) bound non-specially to the proteinaceous factor, (ii) at least one diagnostic label bound to the proteinaceous factor, (iii) Immunoglobulin G bound non-specially to the proteinaceous factor and at least one diagnostic label bound to the proteinaceous factor, and (iv) at least one base supporting the proteinaceous factor.

These and other aspects of the present invention will become apparent to those skilled in the art after a reading of the following description of the preferred embodiment when considered with the drawings.

### BRIEF DESCRIPTION OF THE DRAWINGS

FIG. 1A is a schematic of a device including a base supporting proteinaceous factor according to an embodiment of the present invention;

FIG. 1B is a schematic of a device including a proteinaceous factor conjugated to a diagnostic label according to an embodiment of the present invention;

FIG. 1C is a schematic of a device including proteinaceous a factor conjugated to a diagnostic label and bound to the F_{C} region of Immunoglobulin G (IgG) antibody according to an embodiment of the present invention;

FIG. 1D is a schematic of a device including a proteinaceous factor conjugated to a diagnostic label wherein the diagnostic label involves a chemical conjugate acting on a substrate according to an embodiment of the present invention;

FIG. 1E is a schematic of a device including a proteinaceous factor conjugated to a diagnostic label and bound to the F_{C} region of an IgG antibody according to an embodiment of the present invention wherein the diagnostic label involves a chemical conjugate acting on a substrate;

FIG. 2A is a schematic of a device including a column containing a base supporting proteinaceous factor according to an embodiment of the present invention.

FIG. 2B is a schematic showing a complex mixture of proteins being poured through the device of FIG. 2A and a selective binding of IgG antibody to a proteinaceous factor supported by the base;

FIG. 2C is a schematic showing a washing of non-binding components of the complex mixture of proteins off the column of FIG. 2B;

FIG. 2D is a schematic showing an elution of the IgG antibody selectively bound to a proteinaceous factor to obtain purified IgG antibody.

FIG. 2E shows a detail of a portion of the schematics of FIG. 2C and FIG. 2D showing the binding of a proteinaceous factor to the F_{C} region of IgG antibodies;

FIG. 3A is a schematic of a plurality of antigens;

FIG. 3B is a schematic showing the binding of antigen specific IgG antibodies to the corresponding antigens of FIG. 3A;

FIG. 3C is a schematic showing the a device of FIG. 1B, which includes a diagnostic label, bound to F_{C} region of the antigen specific IgG antibodies of FIG. 3B;

FIG. 4A is a schematic showing the a device of FIGURE ID, which includes as diagnostic label having a substrate and chemical conjugate for acting thereon, bound to F_{C} the region of the antigen specific IgG antibodies of FIG. 3B;

FIG. 4B is a schematic showing the a device of FIGURE ID after the chemical conjugate has acted on the substrate of FIG. 4A; and

FIG. 5 is a graph for the mass determination of Protein V where A) ‘Pure’ Protein V sample demonstrating a major peak at about 60.1 kDa and a minor peak at about 44.5 kDa. B) After internal calibration with b-lactoglobulin (18.3 kDa), bovine serum albumin (66.4 kDa), chicken conalbumin (77.5 kDa) and bovine IgG (147.3kDa), the mass of Protein V was determined to be about 60.1kDa. The “*” represents multicharged species of protein standards.

### DESCRIPTION OF THE PREFERRED EMBODIMENT

In the following description, like reference characters designate like or corresponding parts throughout the several views. Also in the following description, it is to be understood that such terms as “forward,” “rearward,” “left,” “right,” “upwardly,” “downwardly,” and the like are words of convenience and are not to be construed as limiting terms.

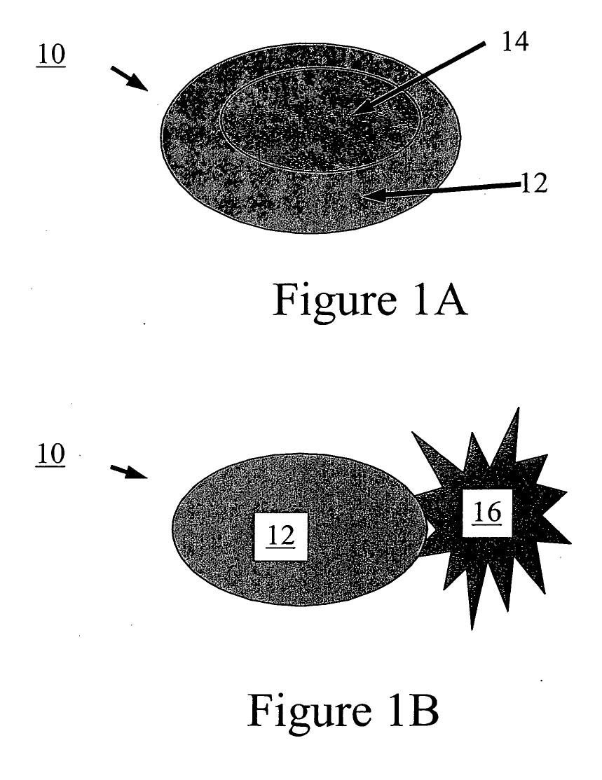

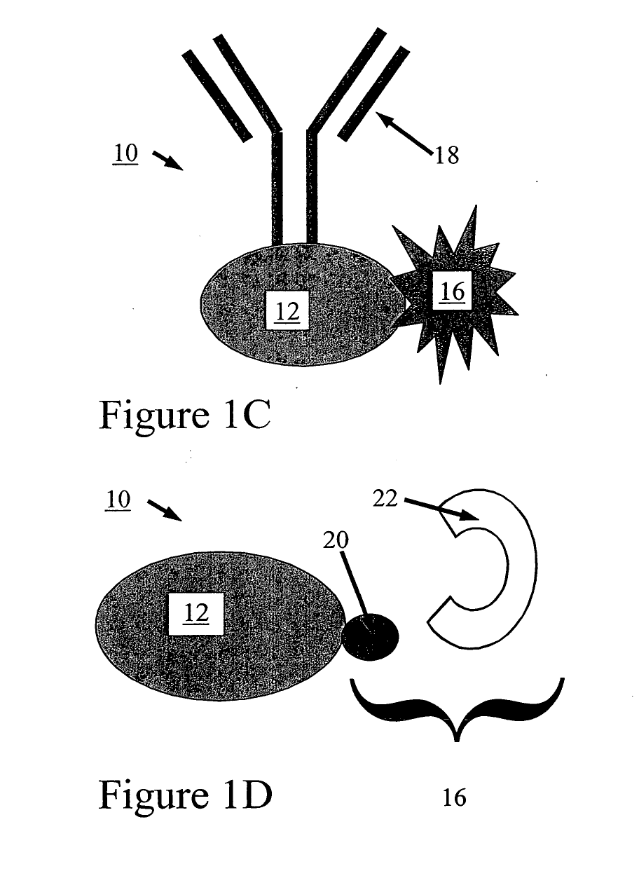

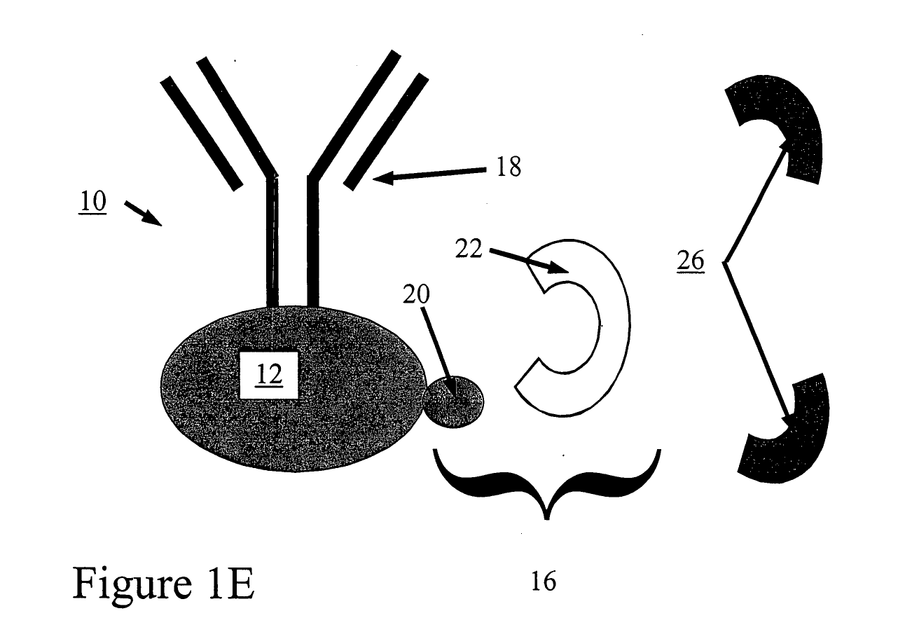

Referring now to the drawings in general and FIGS. 1A through 4B in particular, it will be understood that the illustrations are for the purpose of describing preferred embodiments of the invention and are not intended to limit the invention thereto. As best seen in FIGS. 1A through 1E, a device, generally designated 10, is shown constructed according to the present invention. The device 10 includes a proteinaceous factor 12 encoded by the nucleotide sequence of any one of SEQ ID NO.: 1, a degenerate variant of SEQ ID NO.: 1, and a complement of SEQ ID NO.: 1. The proteinaceous factor 12 may be a recombinant. In addition, the device 10 may include any one of (i) Immunoglobulin G (IgG) bound 18 non-specially to the proteinaceous factor 12, (ii) at least one diagnostic label 16 bound to the proteinaceous factor, (iii) Immunoglobulin G bound 18 non-specially to the proteinaceous factor 12 and at least one diagnostic label 16 bound to the proteinaceous factor 12, and (iv) at least one base 14 supporting the proteinaceous factor 12.

Non-limiting examples of the types of devices 10 contemplated by the applicant include any one of an enzyme immuno assay, an electro-immuno blot, a dot blot, an antibody isolator, an antibody purifier, an antibody isolator and purifier.

Numerous applications for such a device 10 will be apparent to those skilled in the art and may include those disclosed in Volumes 1 and 2 of “Bacterial Immunoglobulin-Binding Proteins: Microbiology, Chemistry, and Biology,” edited by Michael D. P. Boyle, Academic Press, Inc., Harcourt Brace Jovanovich, Publishers, New York, 1990 and “Discovering Genomics, Proteomics, & Bioinformatics,” A Malcolm Campbell and Laurie J. Heyer, CSHL Press, Benjamin Cummings, New York 2003, the disclosure of each is herein incorporated by reference in it entirety, such as, for example, radiolabel bacterial F_{act}-binding proteins as tracers for soluble antigens, assays using enzyme-labeled F_{C}-binding proteins (e.g., a direct binding assay for the detection of IgG antibody to a given antigen and a competitive binding assay for the detection of soluble antigen), detection of specific antibodies, the use of fluorescent-conjugated bacterial immunoglobulin-binding proteins (e.g., fourescein isothiocynate (FITC), tetramethylrhodamine isothiocynate (TRITC), 5 and 6 carboxyrhodamine insothiocyanate (XRITC), and phycobiliprotiens including R-phycoerythrin, allophycocyanin and phycocyanins), boitinylated IgG-binding proteins, immunoelectomicroscopy (e.g., using any on of colloidal gold, silver, ferritin and combination thereof), bacterial F_{C}-binding proteins as probes for antigen-antibody complexes immobilized on membranes (e.g., Western blot analysiso, use of bacterial-bound IgG-binding proteins to analyze labeled antigens, use of bacterial-bound IgG-binding proteins in place of second antibodies for radioimmunoassay, use of bacterial-bound IgG-binding proteins for detection of specific antibodies, depletion of IgG from serum to facilitate measurement of isotypes an antibody other than IgG, use if bacteria expressing immunoglobulin-binding proteins in coagulation assays, use of whole bacteria expressing IgG-binding proteins to detect surface antigens, use of immobilized protein V to purify immunoglobulins, purification and quantification of monoclonal antibodies by affinity chromatography with immobilized Protein V, using immobilized Protein V to isolate IgG, and activating and differentiating human lymphocytes by bacterial F_{C}-binding proteins.

Referring now to FIG. 1A that depicts the device 10 including a base 14 for supporting the proteinaceous factor 12. The base 14 may be an inert solid support such as any one of a polymer, a glass or even a paper. Such inert solid support may comprise a plurality of wells. Non-limiting examples of inert solid supports include any one of a polymer, a glass, a paper and combination thereof. Non-limiting examples of inert solid supports useable as a base 14 for an enzyme immuno assay include polymers such as polystyrene (PS), polyethylene (PE) that may include low-density polyethylene (LDPE) and high-density polyethylene (HDPE), a polypropylene (PP), polyethylene terephthalate (PET), and polyethylene terephthalate glycolate (PETG). Non-limiting examples of inert solid supports useable as a base 14 for any one of an electro-immuno blot and a dot blot include a cellulosic membrane such as a nitrocellulose. Non-limiting examples of inert solid supports useable as a base 14 for any one of an antibody isolator; an antibody purifier; and antibody isolator and purifier include a microbead and a porous membrane.

Referring now to FIG. 1B that depicts the device 10 including proteinaceous factor 12 conjugated to a diagnostic label 16. Non-limiting examples of the diagnostic label 16 include any one of ferritin, gold, silver, a chemical conjugate, a radioactive component and combination thereof. Non-limiting examples of diagnostic labels including a radioactive component may include any one of ³³P, ³H, ³⁵S, ¹²¹I, ¹³¹I, ³²P, ⁵⁴CR, and combination thereof.

Referring now to FIG. 1D that depicts the device 10 including proteinaceous factor 12 conjugated to a diagnostic label 16 including a chemical conjugate 20 such as, for example, an enzyme conjugate. Non-limiting examples of enzyme conjugates include one of a horseradish peroxidase (HRP), an alkaline phosphatase (APAAP), a lactoperoxidase (LPO), a glucose oxidase, digoxigenin and combinations thereof. The chemical conjugate 20 acts on a substrate 22 resulting in cleaved substrate 26. Non-limiting examples of a substrate 22 include those disclosed in the Pierce: Pierce Endogen 2001-2002 catalog, the subject matter of which is herein incorporated by reference, such as, for example, SuperSignal® ELISA Femto and SuperSignal® Pica for ELISA formulations; North2South® fornucleic acid blotting formulation; Lumi-Phos™ WB substrate, SuperSignal® ELISA Femto, SuperSignal® Pica and SuperSignal® West Dura for Western blotting; IPTG, ONPG, and X-Gal for β-galactosidase; INT dye and PMS for glucose oxidase, luciferin for luciferase; ABTS and AEC for peroxidase; and BCIP, Fast Red TR/AS-MX, Lumi-Phos™ WB chemiluminescent substrate, NBT, NBT/BCIP substrate. TMP and PNPP for phosphatase. The cleaved substrate 26 may result in staining by means of a dye or chemiluminescence. Non-limiting examples of chemiluminescent substrates include those disclosed in the Pierce: Pierce Endogen 2001-2002 catalog, such as QuantaBlu™ fluorogenic peroxidase substrate, QuantaBlu™ NS/K substrate and SuperSignal® West HisProbe™.

Referring now to FIG. 1C that depicts the device 10 including Immunoglobulin G bound 18 non-specially to the proteinaceous factor 12 and at least one diagnostic label 16 bound to the proteinaceous factor 12. In the device 10 including Immunoglobulin G (IgG) 18 bound non-specially to the proteinaceous factor 12, the Immunoglobulin G (IgG) 18 is selected for its antigenic specificity. Non-limiting examples of such antigenic specific Immunoglobulin G (IgG) 18 include any one of human IgG, which may be any one of human IgG₁, human IgG₂, human IgG₃, human IgG₄ and combination thereof, horse IgG, bovine IgG, rat IgG, swine IgG, mouse IgG, sheep IgG, goat IgG, guinea pig IgG, hamster IgG, and combinations thereof. Referring now to FIG. 1 E that depicts the device 10 including Immunoglobulin G bound 18 non-specially to the proteinaceous factor 12 and at least one diagnostic label 16 including a chemical conjugate 20 such as, for example, an enzyme conjugate, bound to the proteinaceous factor 12. The operation of the diagnostic label 16 including a chemical conjugate 20 of this device 10 has been described with reference to FIG. 1D.

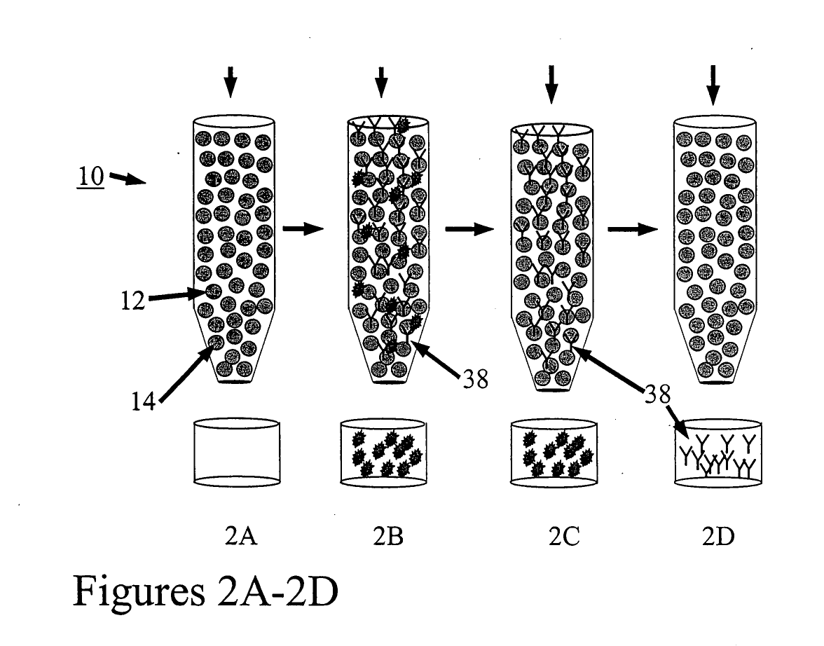

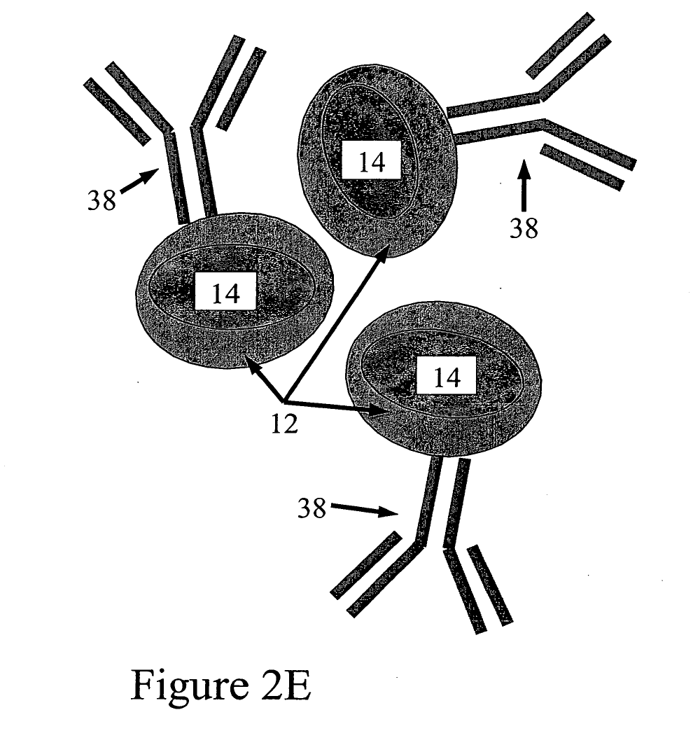

Referring now to FIGS. 2A-2D there is depicted a device 10 for the purification of antibodies. The antibody purification device includes a plurality of device 10 as depicted in FIG. 1A. There is base 14 for supporting the proteinaceous factor 12. The schematics in FIGS. 2A-2D depict a column containing a plurality of microbeads supporting the proteinaceous factor 12 that may be, for example, either Protein V or recombinant Protein V. As depicted in FIG. 2B, a complex mixture of proteins is added to the column. As the mixture passes through the column, selective binding of IgG 38 to the proteinaceous factor 12 separates the IgG 38 from the mixture. As depicted in FIG. 2C, non-binding components of protein mixture are washed out of the column, leaving behind the IgG 38 bound to the proteinaceous factor 12. FIG. 2E is an enlargement of the IgG 38 bound to the proteinaceous factor 12. An elution of purified IgG antibody is depicted in FIG. 2D. This purified IgG antibody can be used in a vast array of molecular biology reactions and assays.

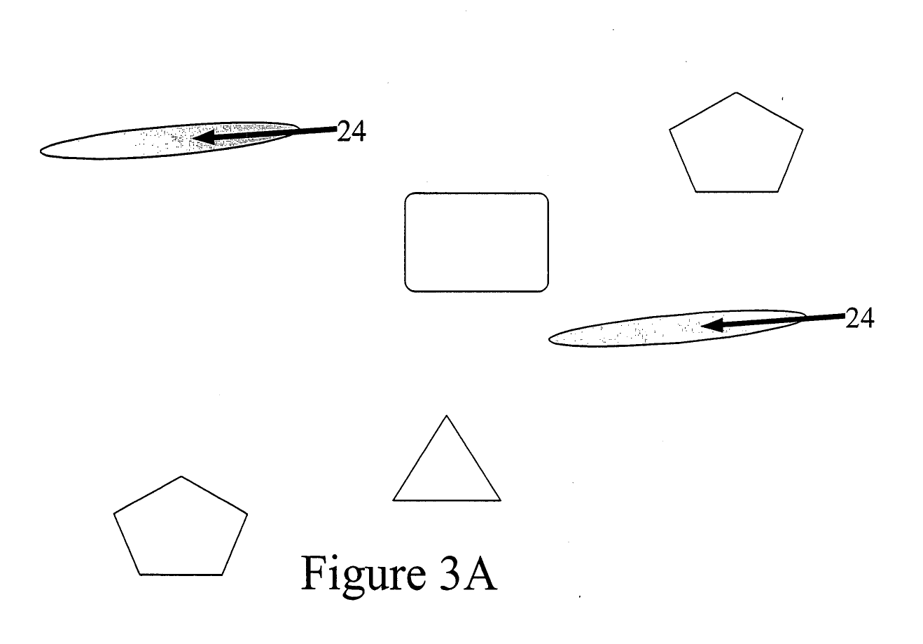

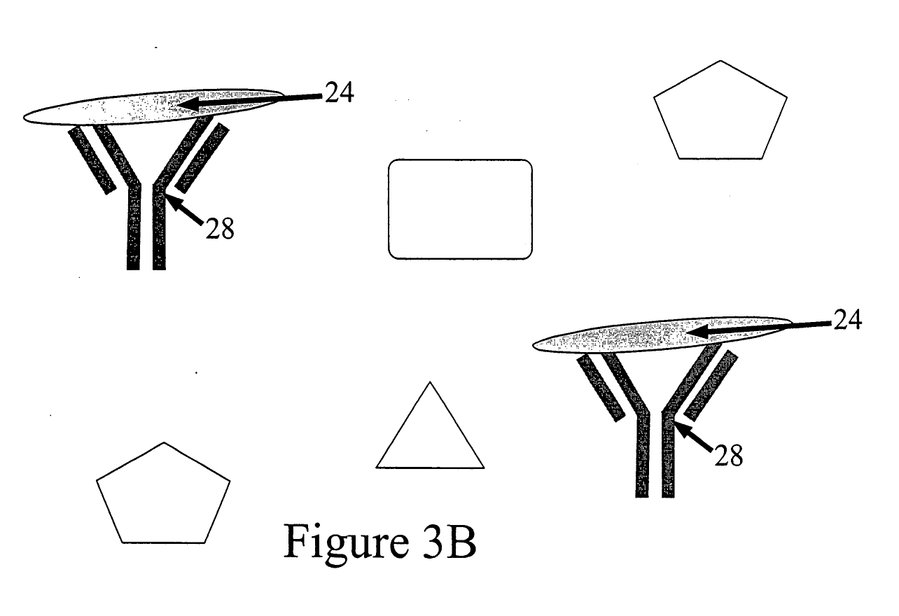

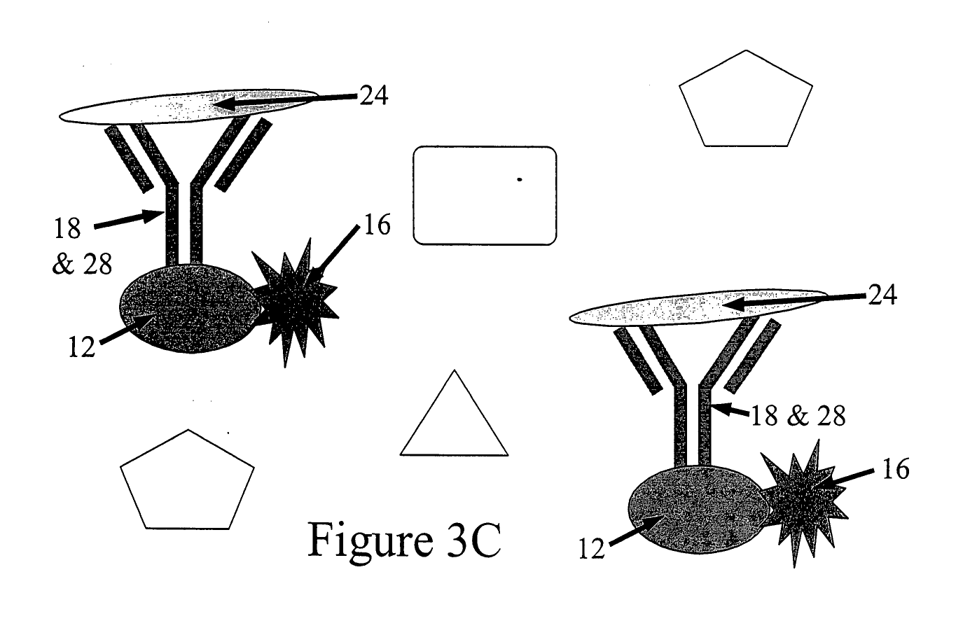

Referring now to FIGS. 3A-3C there is depicted the operation of a device 10 of either FIG. 1B and 1C. FIG. 3A is a schematic of a plurality of antigens. Like antigens have like shapes. In this example, the oval shaped antigen 24 is of interest. To identify the presence of antigens 24, a plurality of anti-antigen 24 antibodies 28 is introduced. As shown in FIG. 3B, antibodies 28 binds specifically to the antigen 24. Thereafter, devices 10 of FIG. 1B including a proteinaceous factor 12 and a diagnostic label 16 are introduced. The proteinaceous factor 12 binds non-specifically to the antibody 28, thereby allowing the identification of the antigen 24 through the presence of the diagnostic label 16 as shown in FIG. 3C.

Alternatively, FIGS. 3A and 3C may be used to demonstrate the operation of a device 10 depicted in FIG. 1C. Such a device 10 includes anti-antigen 24 Immunoglobulin G 18 bound non-specially to the proteinaceous factor 12 and at least one diagnostic label 16 bound to the proteinaceous factor 12. The specific binding of the anti-antigen 24 IgG 18 bound non-specially proteinaceous factor 12 allows the identification of the antigen 24 through the presence of the diagnostic label 16 bound to the proteinaceous factor 12 as shown in FIG. 3C.

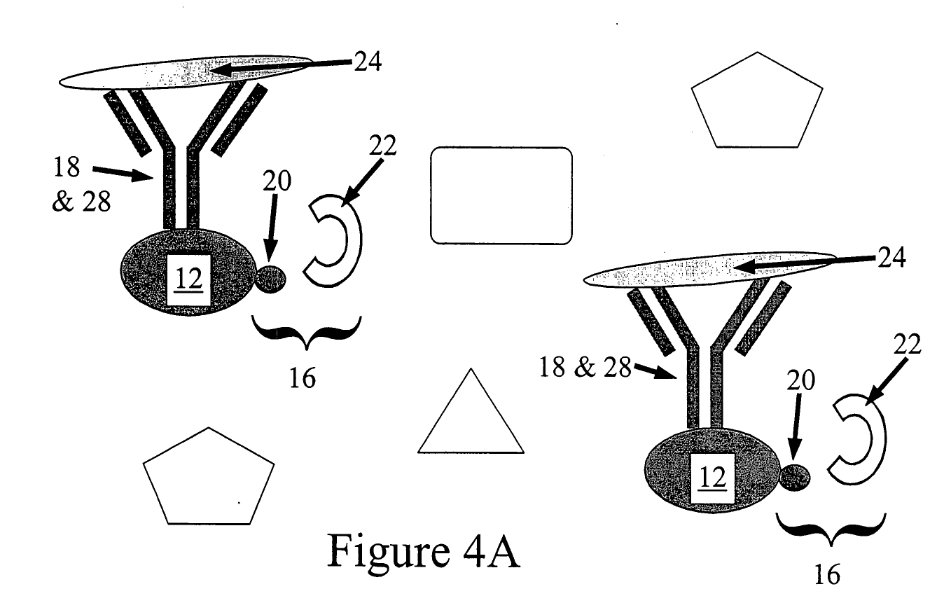

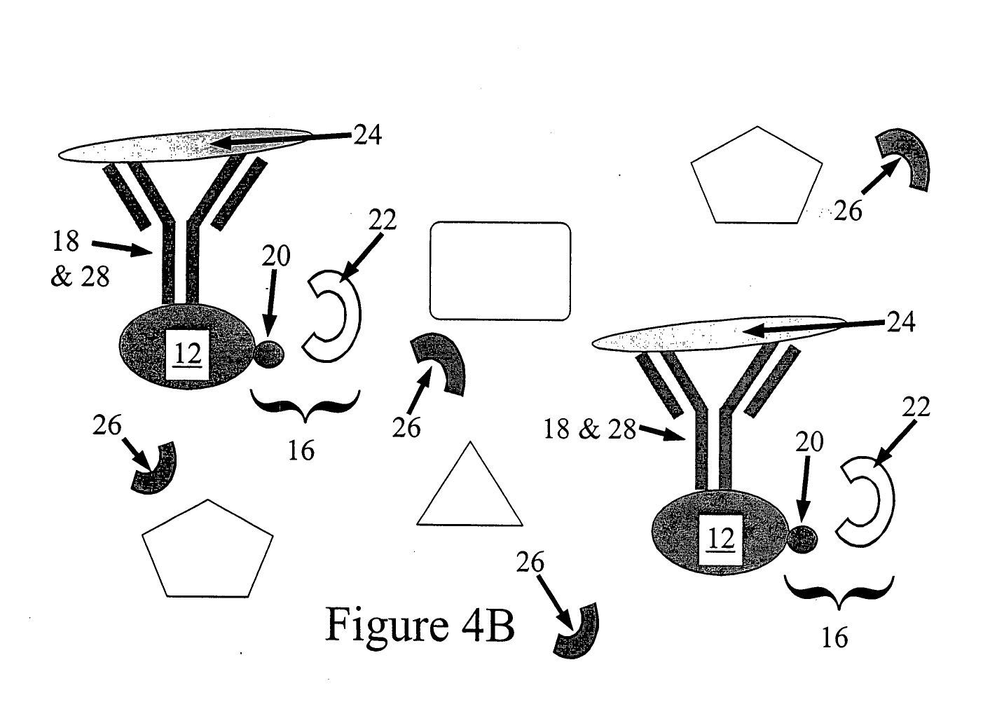

Referring now to FIGS. 4A and 4B in combination with FIGS. 3A and 3B, there is depicted the operation of a device 10 of either FIG. 1D and 1E. As stated above, FIG. 3A is a schematic of a plurality of antigens. Like antigens have like shapes. In this example, the oval shaped antigen 24 is of interest. To identify the presence of antigens 24, a plurality of anti-antigen 24 and antibodies 28 is introduced. As shown in FIG. 3B, antibodies 28 bind specifically to the antigen 24. Thereafter, devices 10 of FIG. 1D including a proteinaceous factor 12 and a diagnostic label 16 including a chemical conjugate 20 are introduced. The proteinaceous factor 12 binds non-specifically to the antibody 28, thereby allowing the identification of the antigen 24 as shown in FIG. 4A through the introduction of a substrate 22 of the diagnostic label 16 to be acted upon by the chemical conjugate 20 to produce cleaved substrate 26 that is detectable as shown in FIG. 4B.

Alternatively, FIGS. 4A and 4B in combination with FIGS. 3A and 3B may be used to demonstrate the operation of a device 10 depicted in FIG. 1E. Such a device 10 includes anti-antigen 24 Immunoglobulin G 18 bound non-specially to the proteinaceous factor 12 and at least one diagnostic label 16 bound to the proteinaceous factor 12. The specific binding of the anti-antigen 24 IgG 18 bound non-specially proteinaceous factor 12 allows the identification of the antigen 24 as shown in FIG. 4A through the introduction of a substrate 22 of the diagnostic label 16 to be acted upon by the chemical conjugate 20 to produce cleaved substrate 26 that is detectable as shown in FIG. 4B.

Protein V is a bacterial protein that binds to mammalian antibody. The U.S. Patent and Trademark Office issued Pat. No. 5,128,451 (the subject mater of which is incorporated herein by reference in it entirety) for a proteinaceous factor based on Protein V and its native organism.

Protein V may be used to isolate and purify those antibodies, which can lead to accurate diagnosis of disease. Also, the isolated antibodies can be used to study the mechanisms of the disease. Protein V may allow for the isolation and purification of mammalian compounds called Antibodies. Protein V is a cell-surface protein from a bacterium and is one of a class of unique proteins, which bind selectively and with high affinity to mammalian compounds called antibodies, which are integral to the mammalian immune system. Protein V may be useful in both the diagnosis of infectious disease and in studies of immune systems.

Protein V represents an excellent example of a biotechnological device. That is, the industrial exploitation of compounds or components from living systems. This technology is especially attractive to Medical diagnostic companies. Protein V represents a potentially significant improvement to the technology currently in use. With improved technology, companies may be more efficient, effective and profitable.

Protein V may be isolated from specific strains of Gardnerella vaginalis. The term “G. vaginalis”, as used herein, is intended to encompass both Haemoohilus vaginalis and Corynebacterium vaginale, in accordance with currently accepted usage. See D. Yong and J. Thompson, J. Clin. Microbiol. 16: 30-33 (1982); see also P. Piot et al., J. Gen. Microbiol. 119: 373-396 (1980).

Protein V is also obtained from certain unclassified coryneform organisms morphologically resembling G. vaginalis. The unclassified coryneform organisms (UCOs) that are the source Protein V are catalase-negative bacteria morphologically resembling G. vaginalis, but are not beta-hemolytic on human blood agar. See P. Piot et al., J. Clin Microbiol. 15: 19-24 (1982). They may be specifically identified as Unclassified Coryneform Organisms of Taxon Cluster 9 in the classification of P. Piot et al., J. Gen. Microbiol. 119; 373-396 (1980). In the classification of P. Piot et al., G. vaginalis is identified as belonging to Taxon Cluster 8. The classification of these organisms is not entirely settled. See D. Yong and J. Thompson, supra; see also Bailey & Scott's Diagnostic Microbiology. 575-587 (E. Baron and S. Finegold Eds., 8th Ed. 1990)(C. V. Mosby Co., St. Louis, Mo.). For present purposes, both G. vaginalis of Piot's taxon cluster 8 and UCO's of Piot's taxon cluster 9 will be referred to herein simply as “G. vaginalis” unless, from the context in which the terms are used, it is apparent that these two groups are being defined separately. Particularly preferred for carrying out the present invention is the strain of Piot's taxon cluster 9 designated as Strain No. AB107 herein, and strains having the identifying characteristics of Strain No. AB 107. Strain No. AB 107 has been deposited with the American Type Culture collection as discussed below.

In operation, the present invention also provides methods for isolating and purifying Protein V from suitable bacteria. Protein V can be solubilized from suitable bacteria, or crude fragments of suitable bacteria, with common reagents including SDS, mutanolysin and cyanogen bromide/HCl. Thus, a variety of extraction procedures are applicable for isolating Protein V, including treatment of whole cells with sodium dodecyl sulfate, aqueous HCl/cyanogen bromide, and mutanolysin. A suitable cyanogen bromide extraction is shown in U.S. Pat. No. 4,945,157 to Boyle and Faulmann and other suitable extraction procedures are given in U.S. Pat. No. 4,883,754 (applicants specifically intend that the disclosure of this and all other patent references cited herein be incorporated herein by reference).

Mutanolysin and aqueous HCl/cyanogen bromide extracts of Protein V are further purified with anion-exchange, high performance, and liquid chromatography. The appropriate peak can be identified by its ability to bind IgG, or as described in the Experimental section below. The peak containing Protein V, when concentrated and applied to an electrophoretic gel and Western blot, shows affinity for non-specific antibody. Crude extracts of Protein V are visualized on SDS-PAGE gels and transfer to nitrocellulose membranes.

Thus, the present invention for isolating Protein V comprises (a) lysing suitable bacterial cells; (b) extracting the lysate with a suitable reagent (e.g., one selected from the group of mutanolysin and aqueous HCl/cyanogen bromide); (c) purifying the crude extract by anion-exchange, high performance liquid chromatography or alternatively extracting the lysate with sodium dodecyl sulfate; (d) further purifying the extract by electrophoresis; and (e) isolating the proteinaceous factor resolving at about 60,000 to about 96,000 daltons.

Protein V of the present invention can also be purified by affinity chromatography on an appropriate immobilized IgG, as described in U.S. Pat. No. 4,883,754.

The present invention also provides methods for purifying or detecting human and other mammalian immunoglobulin G. The method comprises mixing the sample from which the immunoglobulin G is to be isolated and purified with a sample containing Protein V and isolating the material bound by the proteinaceous factors of the present invention. Known methods for accomplishing such isolation and purification include immobilizing the proteinaceous factors of the present invention on a solid support, contacting the solid support to a crude preparation containing the immunoglobulin to be purified, and then removing the crude preparation from the solid support Typically, this method is practiced by immobilizing the Protein V on an affinity support in an affinity column, passing a sample containing IgG through the column, and then adding reagents to chemically release the IgG from the immobilized Protein V. Reference can be made to U.S. Pat. No. 3,966,898 to Sjoguist and Sjodin and U.S. Pat. No. 3,995,018 to Sjoguist for various methods of binding IgG with an IgG binding protein. Various embodiments of the foregoing methods can be routinely practiced by those skilled in the art.

Additionally, the proteinaceous factors of the present invention are labeled in order to identify IgG in samples. Accordingly, the proteinaceous factors are labeled with a radioisotope, enzyme or electron dense ligand. Commonly used radioisotopes suitable for the present purposes include .sup.125 I, .sup.131 I, .sup.3 H, .sup.14 C, and .sup.35 S. Suitable, commonly used enzymes include a horseradish peroxidase (HRP), an alkaline phosphatase (APAAP), a lactoperoxidase (LPO), and a glucose oxidase. Suitable, commonly used electron dense ligands include ferritin, gold and horseradish peroxidase. Labelling may be carried out in accordance with procedures known in the art See, e.g., U.S. Pat. No. 4,883,754.

Suitable bacteria within the scope of this invention include those of natural origin and recombinant origin. The production of cloned genes, recombinant nucleotide, vectors, transformed host cells, proteins, and protein fragments by genetic engineering is well known. See, e.g., U.S. Pat. No. 4,912,038 to Schilling at Col. 3 line 26 to Col 14 line 12. As an example, in the present invention, a nucleotide sequence comprising a cloned gene or fragment thereof that codes for the production of Protein V is produced by generating Protein V nucleotide sequences as either a genomic DNA or complementary DNA library. See generally S. Primrose, Principles of Gene Manipulation, 102-109 (3rd ed. 1985) and T Maniatis et al., Molecular Cloning: A Laboratory Manual, 187-246, 270-307 (1982). Small quantities of DNA obtained from library construction and screenings are able to be amplified by PCR technology to produce sufficient quantities for cloning into appropriate vectors. See generally U.S. Pat. No. 4,683,195 to Mullis et al. and U.S. Pat. No. 4,683,195 to Mullis.

The production of suitable bacteria requires construction of expression vectors containing the gene for Protein V operably linked to suitable control sequences capable of effecting the expression of Protein V in suitable host cells. The vectors comprise plasmids, viruses, phage, and integratable DNA fragments (i.e. fragments integratable into the host genome by recombination). Whether the vector replicates and functions independently of the host genome or integrates into the host genome itself, expression of the proteinaceous factor is dependent on regions within the vector that are operably linked or functionally associated with the gene coding for the Protein V, and are operable in the host organism. Such functional regions ordinarily include an origin of replication (if necessary), a promoter located upstream from the DNA encoding the Protein V, an RNA splice site (if intron-containing genomic DNA is used), a polyadenylation site, and a transcriptional termination sequence. If the vector does not contain a viral origin of replication, the mammalian cells may be transformed with a selectable marker, such as dihydrofolate reductase, and the Protein V DNA. This method is further described in U.S. Pat. No. 4,399,216. A broad variety of suitable prokaryotic and eukaryotic vectors are available. For example, an Escherichia coli host is typically transformed using the plasmid pBR322 or its derivative, insect cells are typically transformed with a baculoyirus expression vector such as those derived from Autograghica califomica MNPV, and mammalian cells are generally transformed with vectors containing a MMTV LTR sequence or SV-40 promoter. Such mammalian vectors are generally inaudible with drugs, such a dexamethasone, as well as capable of conferring selectivity to the host cell by containing a gene encoding resistance to other drugs, such as neomycin.

Transformed host cells, which produce the Protein V upon transformation or transfection with the vectors constructed with the gene for Protein V, may be derived from mammalian or insect sources. Propagation of such cells in cell culture has become a routine procedure (Tissue Culture, Academic press, Kruse and Patterson, editors (1973)). Examples of suitable mammalian cells include VERO, HeLa, CHO, WI138, BHK, COS-7, CV, and MDCK cell lines, while insect cells are typically cultured Spodootera fruoigerda described in U.S. Pat. Nos. 4,745,051 and 4,879,236 to Smith et al.

Prokaryotic cells are also excellent hosts, and include gram positive and gram negative organisms. A representative group of suitable hosts include E. coli W3110 (ATCC 27,325), E. coli B, E. coli X1776 (ATCC 31,537) and E. coli 294 (ATCC 31,446).

Eukaryotic yeast cultures may also be transformed with Protein V encoding vectors. See e.g. U.S. Pat. No. 4,745,057. Saccharomyces Cerevisiae is the most commonly used among lower eukaryotic host microorganisms, although a number of other strains are commonly available. Suitable vectors and promoters for the use in yeast expression are further described in R. Hitzeman et al., EPO Publn. No. 73,657. Transformants may be screened for the production of Protein V by any convenient procedure. For example, a method may be to first transfer colonies from an agar plate to nitrocellulose filters, and then use an antibody to Protein V in a colormetric assay to determine which colonies are producing the Protein V Other methods include hybridization selection and in situ hybridization. See generally T. Maniatis et al., supra at 310-352.

As noted above, the present invention provides a method for the production of Protein V from a variety of cell and vector combinations, such as by transforming the host cell with an expression vector containing the gene encoding Protein V. In general, purification of Protein V from these sources comprises culturing a host cell that expresses the Protein V and harvesting the proteinaceous factor from the culture. This culture can be carried out in any suitable fermentation vessel, with a growth media and under conditions appropriate for the expression of the Protein V in the chosen host cell. The Protein V is collected directly from the culture media, or the host cells are lysed and the Protein V collected therefrom. The Protein V is then further purified in accordance with known techniques.

Cloned genes of the present invention may code for Protein V of any species of origin, including bacterial, murine, porcine, bovine, feline, and human, but preferably code for Protein V of bacterial origin. Nucleotide sequences that code for Protein V, or any proteinaceous factor having the characteristics of Protein V, but differ in code sequence from the isolated sequences due to degeneracy in the genetic code, are also an aspect of this invention. The genetic degeneracy is well known in the literature. See, e.g., U.S. Pat. No. 4,757,006 to Toole et al. at Col. 2, Table 1. Therefore, nucleotide sequences which hybridize to DNA that encodes Protein V from G. vaginalis, whether from different species or due to a degeneracy in the genetic code, are aspects of this invention.

In the following experimental section there are set forth examples that illustrate procedures, including the best mode for practicing the present invention In these examples, “nm” means nanometers, “mm” means millimeters, “ng” means nanograms, “mg” means milligrams, “g” means grams, “μl” means microliters, “ml” means milliliters, “mmol” means millimoles, “mM” means milliMolar, “M” means Molar, “G” means gravity, “U” means Units, and temperatures are given in degrees Centigrade unless otherwise indicated.

Experimental

I. Methods

A. Bacteria

Strain No. AB 107 was isolated from a patient with bacterial vaginosis. This strain formed small, grayish, non-hemolytic colonies on human blood tween (HBT) agar and sheep blood agar. It was also catalase negative and hydrolysed starch and hippurate. Strain No. AB 107 cultured either on HBT Agar plates or in Columbia broth supplemented with 5% fetal calf serum under microaerophilic conditions at 37° C. for 48 hrs. Whole cells of Strain No. AB 107 were harvested from broth cultures by centrifugation and washed once with phosphate buffered saline, Ph 7.2 (PBS), and stored at −20° C. until used. Strain No. AB107 was identified according to established morphological and biochemical criteria as belonging the group of unclassified coryneform organisms identified as taxon-cluster 9. See P. Piot et al., J. Clin. Microbiol. 15: 10-24 (1982); p, Piot et al., J. Gen. Microbiol. 119: 373-396 (1980). Strain No. AB 107 has been deposited with the American Type Culture Collection, 12301 Parklawn Dr., Rockville, Md. 20852, USA, in accordance with the provisions of the Budapest Treaty on Jun. 13, 1991, and designated as ATCC Deposit No. 55195.

B. Extractions

1. Mutanolysin Extract Ion

Aliquots containing approximately 0.25 g of bacteria (wet weight) were extracted with Mutanolysin (Sigma Chemical Co., St. Louis, Mo.) by the method of Siegel et al., Infect. Immun. 31: 808-815 (1981), with slight modifications. Specifically, enzyme extraction of the bacterial pellet was done in 6.0 ml of buffer containing 2000 U of mutanolysin in 0.05 M KH₂PO₄, pH 6.5. Extractions were performed for 4 hours at 37° C. The suspension was then centrifuged at 10,000×G for 15 minutes. The supernatant was then dialyzed overnight against PBS at 4° C. The crude extract was then concentrated in a collodion bag (75,000 HM cut-off) (Schleicher and Schuell, Inc., Keene, N H) to a volume of approximately 100 μl and stored at −20° C. until used.

2. Cyanogen Bromide/HCl Extractions

Whole cell pellets (approximately 1.0 g wet Weight) were suspended in 0.1 M HCl with and without reagent grade cyanogen bromide (Pierce Chemical Co., Rockford, Ill.) at a concentration of 15 mg/ml. After slow stirring for 18 hours at room temperature, the suspensions were spun at 10,000×G for 15 minutes. The supernatant was dialyzed against deionized H₂O. The crude extracts were then concentrated in collodion bags to volumes of approximately 100 μl and stored at −20° C. until used.

3. High-Performance Liquid Chromatography of CNBr/HCl or Mutanolysin Crude Extracts of Protein V.

The chromatographic system used for anion exchange purification of CNBr, HCl or mutanolysin extracts of Protein V consisted of model 510 solvent delivery systems, a model 810 B WISP autosampler, a model 490 UV detector set at 280 nm and a model 840 data and chromatography control station (Waters Chromatography Division, Milford, Mass.). A Protein-Pak DEAE-5PW anion exchange column (Waters) was equilibrated with 25 mM TrisHCl, pH 7.5 in pump A before injection of up to 80 μl of sample. Pump B contained 25 mM tris-HCl, pH 7.5 with 1.0 M NaCl. Gradient conditions were set at a 30 minutes linear gradient, 0-100% B at a flow rate of 1.0 ml/minute. Fractions of the various peaks were collected and concentrated in collodion bags and stored at −20° C. until use. Identification of the fraction containing purified Protein V was accomplished by testing each fraction by dot-blot as described below.

4. Sodium Dodecyl Sulfate Extractions.

A bacterial pellet of approximately 0.25 g wet weight was boiled in 1.0 ml of 2% SDS in deionized water for 10 minutes. The suspension was centrifuged and the proteins in the supernatant were precipitated by the addition of 0.5 ml of 30% trichloroacetic acid. The pellet obtained by centrifugation was washed once with ethanol and once with acetone. The remaining pellet was stored at −20° C. until use.

5. HCL Protein Extraction of Bacterial Cells:

Bacterial cells are pelleted and extracted overnight at 40C in 100 ml of 0.1N HCL with stirring. The cells are then centrifuged at 10,000×G for 10 minutes and the supernatant is removed and dialyzed (dialysis in a 10,000 MW cutoff) against 50 mm Phosphate buffer.

C. Immunoylobulins

Human polyclonal IgG subclasses were obtained from the WHO/IUIS Immunoglobulin subcommittee. Polyclonal goat, chicken, rabbit, swine, rabbit and mouse that were conjugated to horse radish peroxidase (HRP) were obtained from Kirkegaard and Perry Laboratories, Inc., Gaithersburg, Md. and from Accurate Chemical Co., Westbury, N.Y.

D. Electrophoresis

All electrophoresis techniques, such as polyacrylamide gel electrophoresis in the presence of sodium dodecyl sulfate (SDS-PAGE) or isoelectric focusing (IEF), were performed with the Phastsystem separation and development unit (Pharmacia, Uppsala, Sweden). See D. Anton and R. Kutny, J. Biol. Chem. 262: 2817-2822 (1987); and I. Olsson et al., Electrophoresis 9:16-22 (1988). SDS-PAGE gels, IEF gels, buffer strips, molecular weight and pI standards, Coomassie Blue and silver staining kits were also obtained from Pharmacia. The dimensions of the SDS-PAGE gels were 50.times.43.times.0.45 mm. The acrylamide concentration was 4% for the stocking gel and 12.5% for the separation gel. The buffer system in the gels was 0.112 M Tris, 0.112 M acetate, pH 6.4. Buffer strips contained 2% agarose, 0.2 M Tris, 0.2 M N-tris (hydroxymethyl) methyl-glycine, pH 8.1 and 0.55% SDS. The size of the IEF gels were 50.times.43.times.0.35 mm. The concentration of acrylamide in IEF the media was 5. The IEF gels contained ampholytes (Pharmalyte, Parmacia) in a pH range of 3-9 or 4-6.5 with a buffering capacity of 0.02 mmol/ml of gel. Samples for SDS-Page were adjusted to concentrations of approximately 100 ng of protein in sample buffer (10 mM Tris-HCl, 1.0 mM EDTA, 2.5% SDS, 5% 2-mercaptoethanol and boiled for 5 minutes). Samples for IEF were adjusted to concentrations of 10-50 ng in deionized H.sub.2 O. Samples were applied to gels in a volume of 1.0 μl. All conditions of separation and staining were controlled by the computerized system as outlined in the manual. The duration of each step was controlled by volt×hours (Vh). The maximum voltage, current and power for IEF gels was 2000 V, 25 mA, and 7 W and for SDS-PAGE gels was 250 V, 10 mA and 3 W. All gels were run at a constant temperature of 15° C. Staining procedures such as Coomassie Brilliant Blue and silver staining were automatically performed in the development unit according to manufacturers instructions and have been described elsewhere. D Anton and R. Kutny, supra; I. Olsson et al., supra.

E. Western Blotting

PhastGel media, being ultra-thin (SDS-PAGE-0.45 mm, IEF-0.35 mm), were particularly suitable for diffusion blotting according to the method of Beisiegel. See I. Olsson et al., supra. Diffusion blotting was performed by placing an Imobilon-NC nitrocellulose membrane (Cat # HAHY 13250, Millipore Corp., Bedford, Mass.), which had been cut to the exact dimensions of the separation gel, on the gel surface. For SDS-PAGE gels the transfer was incubated at 70° C. for 20 minutes. IEF gel transfers were incubated at room temperature for 20 minutes. After transfer, the membranes were place in Milk Diluent/Blocking Solution (Kirkegaard & Perry Laboratories, Inc., Gaithersburg, Md.) for 1.0 hour. The membranes were then washed in 0.02% Tween-20 for 30 minutes. The membranes were probed with Antibody-Horse Radish peroxidase conjugates for 1.0 hour at a dilution of 1:1000 in a washing buffer. When the unconjugated, human Ig subclasses were used as first antibody, a second probing followed with chicken anti-human antibody conjugated to HRP. After probing, the membranes were washed three times for 15 minutes each. The membranes were then developed with a solution of 3,3′,5,5′-Tetramethylbenzidine (TMB membrane peroxidase substrate kit, Kirkegaard & Perry Laboratories, Inc.).

F. Dot Blotting

Whole cells of strain No. AB107 were washed once in PBS and adjusted to a concentration of 1×10⁸ cells/ml the concentration of organisms was standardized by measuring the: Optical/Density at 550 nanometers (OD550). Dot blots were performed by using the Bio-Rad bio-dot microfiltration apparatus (Bio-Rad laboratories, Richmond, Calif.). Nitrocellulose membranes (0.45 um, Bio-Rad) were soaked 20 mM Tris, 500 mM NaCl pH 7.5 (TBS) and placed in the apparatus. Whole cell suspensions (100 μl) were pipetted into the wells. Serial dilutions of bacteria were applied to establish optimal binding conditions. After washing the bactyeris in each well with TBS containing 0.5% Tween 20 (U.S. Biochemical Corp., Cleveland, Ohio), the nitrocellulose was removed and washed thee times, for 15 minutes each time, in 100 ml of TBS-tween 20. The nitrocellulose membrane was then probed and developed as described in the Western blotting procedure.

G. Protein Concentrations

Total protein concentrations were measured with the BCA protein concentration assay (Pierce Chemical Co., Rockford, Ill., USA).

H. Construction and Screening of a Recombinant DNA Library.

Genomic DNA was isolated by a method in Current Protocols in Molecular Biology (John Wiley and Sons, Inc., New York, N.Y.) as described for Gram negative bacteria. The isolated DNA was digested with mechanical shearing and the DNA fragments were processed with Klenow treatment before being fractionated by agarose gel electrophoresis. Fragments in the size range of 2 to 23 kb were excised from the gel, purified by electroelution, and ligated into the Lambda Zap plasmid cloning vector (Strategene, La Jolla, Cailf.). The recombinant plasmids were transformed into E. coli DH5α or XL-1 blue. Transformed cells were plated onto Luria Bertani agar containing 60 μg of Ampicillin (Sigma Chemical, St. Louis, Mo.) and grown at 35° C. overnight. Resulting colonies were analyzed by Western blot analysis with a 1:500 dilution of polyclonal Goat IgG ant-mouse antibody conjugated with horseradish peroxidase. Blots were developed with 3,3′,5,5′-tetramethlbenzidine(KP1, Gaithersburg, Md.). Positive colonies were identified and characterized.

I. Nucleotide Sequence Determination and Analysis.

The complete nucleotide sequences of both strands of the DNA insert in plasmid pBSPV were determined by the dideoxy-chain termination method (Current Protocols in Molecular Biology). The nucleotide sequence and the deduced amino acid sequence were analyzed with the Vector NTI (InfofMax, Inc., Bethesda, Md.) software. Sequence similarity searches were performed with GenBank sequences by using the BLAST network service.

J. Overexpression of the Recombinant Protein.

The DNA insert in pBSPV was cloned in frame into a pET101 TOPO® expression vector (Invitrogen, Carlsbad, Calif.) to create pETPV and was transformed into E. coli TOP10-competent cells and then into E. coli BL21DE3+ cells and overexpressed by following the manufacturer's protocols (i.e., pET Directional TOPO® Expression Kits: Five-minute, directional TOPO® cloning of blunt end PCR products into vectors for high-level, inducible expression in E. coli, Catalog Nos. K100-01, K101-01, K102-01 (Version A, 010124 25-0400 and Version C 032702 25-0400) form Invertrogen™ life technologies, Invertrogen Corporation, the subject matter of each is herein incorporated by reference in its entirety).

K. Molecualr Weight Determination of Protein V with Sensitive, Laser-Induced Time-of-Flight Mass Spectrometer (SELDI-TOF-MS) Based ProtienChip® Technology Form Ciphergen Biosystems, Inc.

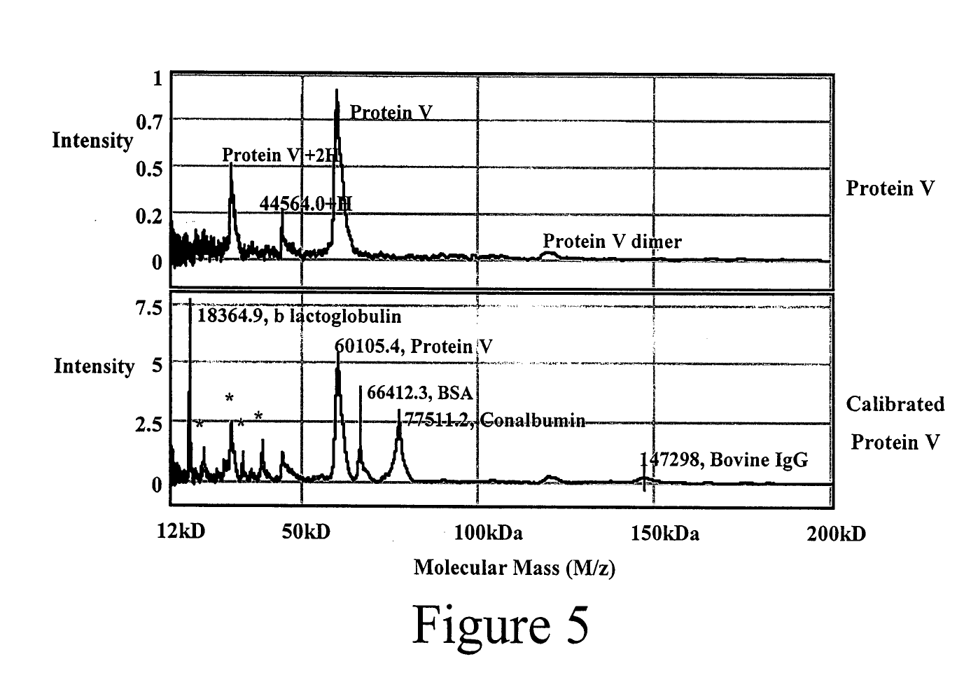

FIG. 5 is a graph for the mass determination of Protein V where A) ‘Pure’ Protein V sample demonstrating a major peak at about 60.1 kDa and a minor peak at about 44.5 kDa. B) After internal calibration with b-lactoglobulin (18.3 kDa), bovine serum albumin (66.4 kDa), chicken conalbumin (77.5 kDa) and bovine IgG (147.3kDa), the mass of Protein V was determined to be about 60.1kDa. The “*” symbol represents multicharged species of protein standards. This graph was generated using SELDI ProteinChip® technology that enables selective protein retention on surfaces by means of distinct chromatographic or bioaffinity surfaces. Once the proteins are bound to the array surface, a set of buffers is used to wash away unbound proteins and other elements of the original sample. Ciphergen's ProteinChip Reader then detects proteins retained on the array surface. The process begins when a laser desorbs and ionizes proteins from the array surface. Once desorbed from the array surface, the ions are accelerated through the flight tube of the ProteinChip Reader and are read by a detector. The velocity at which the ions are accelerated though the flight tube to the detector is a function of mass; smaller ions will reach the detector faster than larger ions. The raw data produced by the TOF-MS-based (time-of-flight mass spectrometer) ProteinChip Reader plots peak intensity against molecular weight. Ciphergen's ProteinChip® Reader enables researchers to identify and differentiate proteins bound to ProteinChip Array surfaces according to their molecular weight. Selective protein retention combined with the ability to assess a protein's molecular weight makes it possible to identify hundreds of unique proteins from a single sample. The SELDI process is covered by U.S. Pat. Nos. 5,719,060; 5,894,063; 6,020,208; 6,027,942; and 6,225,047. Additional US pending applications may relate to the SELDI process including publishd application Nos. 200020060290; 20020138208; 20020182649; and 20030062473. Two publications that demonstrate the use of SELDI ProteinChip® technology include J. C. Howard et al., “Identification of Collagen-Binding Proteins in Lactobacillus spp. with Surface-Enhanced Laser Desorption/Ionization-Time of Flight ProteinChip Technology,” Applied and Environmental Microbiology, October 2000, p. 4396-4400 and J. C. Howard “Rapid Identification of Probiotic Lactobacillus Biosufactant Proteins by ProteinChip Tandem Mass Spectrometry Tryptic Peptide Sequencing,” Applied and Environmental Microbiology, February 2002, p. 977-980. The disclosure of these patents, patent publications and publications are hereby herein incorporated by reference in their entirety.

II. Results

Whole cell suspensions of strain #AB107, when bound to nitrocellulose membranes and probed with horseradish peroxidase conjugates of various non- specific immunoglobulins in the manner described above, demonstrate strong affinity for Human IgG-F_{C} fragments, goat IgG and swine IgG whole antibody as shown in FIG. 7. No affinity was observed for Human IgM whole antibody. Protein V demonstrates weak affinity for Human IgG-F(ab′)₂ fragments.

The foregoing is illustrative of the present invention, and not to be construed as limiting thereof. The invention is defined by the following claims, with equivalents of the claims to be included therein.

Certain modifications and improvements will occur to those skilled in the art upon a reading of the foregoing description. It should be understood that all such modifications and improvements have been deleted herein for the sake of conciseness and readability but are properly within the scope of the following Claims.

1-115. (canceled)

116. A method for making a recombinant proteinaceous factor usable alone or in a device, the method comprising transforming a vector including any one of SEQ ID NO.: 1, a degenerate variant of SEQ ID NO.: 1, a complement of SEQ ID NO.: 1, or any combination of any of the preceding into a host to express the recombinant proteinaceous factor encoded thereby.

117. (canceled)

118. The method according to claim 116, wherein the transforming a vector comprises transforming a vector comprising SEQ ID NO.: 1 into a host to express the recombinant proteinaceous factor encoded thereby.

119. The method according to claim 116, wherein the transforming a vector comprises transforming a vector comprising a degenerate variant of SEQ ID NO.: 1 into a host to express the recombinant proteinaceous factor encoded thereby.

120. The method according to claim 116, wherein the transforming a vector comprises transforming a vector comprising a complement of SEQ ID NO.: 1 into a host to express the recombinant proteinaceous factor encoded thereby.

121. The method according to claim 116, wherein the host comprises any one of a mammalian cell, a prokaryotic cell, or an insect cell.

122. The method according to claim 121, wherein the mammalian cell comprises any one of a VERO cell line, a HeLa cell line, a CHO cell line, a WI138 cell line, a BHK cell line, a COS-7 cell line, a CV cell line, aMDCK cell line, or any combination of any of the preceding.

123. The method according to claim 121, wherein the insect cell comprises a cultured Spodootera fruoigerda.

124. The method according to claim 121, wherein the prokaryotic cell comprises any one of a gram positive organism, gram negative organism, or any combination of any of the preceding.

125. The method according to claim 121, wherein the prokaryotic cell comprises any one of an E. coli W3110 (ATCC 27,325), an E. coli B, an E. coli X1776 (ATCC 31,537), an E. coli 294 (ATCC 31,446), an E. coli TOP10, an E. coli BL21DE3+, or any combination of any of the preceding.

126. The method according to claim 121, wherein the prokaryotic cell comprises any one of an E. coli TOP10, an E. coli BL21DE3+, or any combination of any of the preceding.

127. The method according to claim 121, wherein the prokaryotic cell comprises an E. coli TOP10 and an E. coli BL21DE3+.

128. The method according to claim 116, wherein the host comprises a eukaryotic host microorganism.

129. The method according to claim 128, wherein the eukaryotic host microorganism comprises a lower eukaryotic host microorganisms.

130. The method according to claim 126, wherein the eukaryotic host microorganism comprises a Saccharomyces cerevisiae.


SEQUENCE LISTING (2 sequences)

SEQ ID NO: 1 - DNA, 1569 bp, Gardnerella vaginalis Taxon Cluster 8
  CDS (1)..(1566)
atgaaaagagaaagattagataagttttcaatcagaaagttaactgtgggagcagcttcagtattgattggtgtagcatttggtgctacaactgttagtgcagcaccaactgtaccacaagctcctgcaacaggtcaacaagctgcagaagttactgaacttaaggacgttaagttcactttcaagatgggtgataagtcagctactactgagccagtaacagtttatgttactaaagatggtcactacggttttgaaaatgacgctgctaagtgtaaatctactgcagctcagattgtttcagctttcttggataaagtaactgacaatgatattaaaaatgccgaaggtgtagcttatccagaaaagattaacgctggtgagaacactgttgaggtagctgctaaacctgcttctaacaaagtagaacatagtgatgctgatttgcaaaaagaagcagaaaaaaaggctgatgaacttacgaacagtcctgcttttttcaaggctgagttaaaagatgcagaacatgacttgcaagtagctaaagatacagttgcacaagcagagcaagaaggcagcttgtcacaagaagaaatggctaaacttcaagatgacgttgcaaaggcagaagcaaaggtcaacgaacttaagaacagttctgcttttgttgaagttcagttaaaagatgcagagcatgaattggaagtagctaaagatgcagttgcacaagcagagcaagaaggtggcttgtcacaagaagaaatgggcaagcttcaagatgaagttgcaaaggcagaagcagaagttaacgaattgaaagcaaaacttgcaaaaaaggtaactccagcaccagaaccagtaaaaccaactccagtagttccaacccctgaaccaaaaccagaagaaacaacttaccacttcacatacaaaggtgcagatactactgtaagcacagatcacgtagcaggaagtgttgacaaagcagaacaatacttcagagcatatgctagtgaatcaggtcttaatttagactttacatacgatgaagctacacatacttttgtaggtactgatgctaaacctgcaaaaactgtttaccacttccaatacgatgacaagaagggtaacagcattagacaagattttgcaacaacaagtaaagatacagcagaaatgcacttcagagcatatgctagcgataatgcactctccatagatgatgcacactttacatacgatgaagctacacatacatttgtatacaaagacttcgaatctgagaagggtgaaccagaagtacaaccagaagcaccagtgcaaaagattgttttccacttccaatacgatgacaagaagggtacaagcattagacaagatttcgcagcagtaaataaagaaattgcagaaatgcacttcaaagaatatgctactgaaagtggacttgtattagatgatgcacactttgcatacaatgaagctaaccaaacatttgtatacaaggacttcgagtcagagaagggtgaaccagaagtattgccagcaccagcagttgatgtgacttgcagaagc

SEQ ID NO: 2 - protein, 522 aa, Gardnerella vaginalis Taxon Cluster 8
MKRERLDKFSIRKLTVGAASVLIGVAFGATTVSAAPTVPQAPATGQQAAEVTELKDVKFTFKMGDKSATTEPVTVYVTKDGHYGFENDAAKCKSTAAQIVSAFLDKVTDNDIKNAEGVAYPEKINAGENTVEVAAKPASNKVEHSDADLQKEAEKKADELTNSPAFFKAELKDAEHDLQVAKDTVAQAEQEGSLSQEEMAKLQDDVAKAEAKVNELKNSSAFVEVQLKDAEHELEVAKDAVAQAEQEGGLSQEEMGKLQDEVAKAEAEVNELKAKLAKKVTPAPEPVKPTPVVPTPEPKPEETTYHFTYKGADTTVSTDHVAGSVDKAEQYFRAYASESGLNLDFTYDEATHTFVGTDAKPAKTVYHFQYDDKKGNSIRQDFATTSKDTAEMHFRAYASDNALSIDDAHFTYDEATHTFVYKDFESEKGEPEVQPEAPVQKIVFHFQYDDKKGTSIRQDFAAVNKEIAEMHFKEYATESGLVLDDAHFAYNEANQTFVYKDFESEKGEPEVLPAPAVDVTCR